(19) United States
(12) Patent Application Publication
(10) Pub. No.: US 2010/0267840 A1
(43) Pub. Date: Oct. 21, 2010

(54) STABILIZERS FOR HYDROPHOBIC COMPONENTS IN PERSONAL CARE COMPOSITIONS

(76) Inventors: Susan L. Jordan, Doylestown, PA (US); Xiaodong Zhang, Livingston, NJ (US); J. Keith Harris, Midland, MI (US); Edward F. DiAntonio, Staten Island, NY (US); Gene D. Rose, Alexandria, MN (US); Linda A. Kosensky, Manville, NJ (US)

Correspondence Address: The Dow Chemical Company, P.O. BOX 1967, Midland, MI, 48641, US

(21) Appl. No.: 12/665,248

(22) PCT Filed: Jun. 20, 2008
(86) PCT No.: PCT/US08/67600

§ 371 Date: Jun. 21, 2010

Related U.S. Application Data
(60) Provisional application No. 60/945,420, filed on Jun. 21, 2007.

(51) Int. Cl. A61K 8/90

(52) U.S. Cl. 514/725

(57) ABSTRACT

The present invention provides personal care compositions, as well as methods for stabilizing a degradable of reactive hydrophobic, cosmetically acceptable, component in an aqueous composition.

### FIELD

The present invention relates to personal care compositions.

### BACKGROUND

Many personal care compositions contain water insoluble actives. Such actives may exhibit a tendency to degrade in the formulation. For example, Vitamin A (retinol) is photolabile. While provitamins can be used in place of such a component, they are typically less efficacious. In another example, fragrances can evaporate, thereby decreasing consumer satisfaction. In yet another example of the difficulties of incorporating hydrophobic actives into personal care compositions, certain flavors can destabilize oral care formulations.

One strategy to overcome such difficulties is to encapsulate the hydrophobic active. In the past, encapsulation has required high shear and/or solvent exchange processes.

Thus, what is needed are improved compositions containing stabilized hydrophobic personal care actives, and methods for producing the same.

### SUMMARY

In one embodiment, the present invention provides personal care compositions, comprising a hydrophobic, cosmetically acceptable, component comprising at least one of a personal care active, vitamin, dye, flavor, or fragrance; and a block polymer comprising at least one block of polymerized ethylene oxide and at least one block of a polymerized butylene oxide.

In another embodiment, the present invention provides methods for stabilizing a degradable or reactive hydrophobic, cosmetically acceptable, component in an aqueous composition, comprising encapsulating a percentage of the hydrophobic component in a block polymer having at least one block of polymerized ethylene oxide and at least one block of polymerized butylene oxide.

### DETAILED DESCRIPTION

In one embodiment, the present invention provides personal care compositions, comprising a hydrophobic, cosmetically acceptable, component comprising at least one of a personal care active, vitamin, dye, flavor, or fragrance; and an ethylene oxide/butylene oxide block polymer.

In a preferred embodiment, the block polymer comprises at least one block of polymerized ethylene oxide having about 4 to about 16 ethylene oxide (“EO”) units and at least one block of polymerized butylene oxide having about 4 to about 40 butylene oxide (“BO”) units. The BO unit refers to 1,2-butylene oxide. It is understood that the number of EO and BO units can be conventionally calculated by using NMR to get the number average molecular weight of each block of ethylene oxide and butylene oxide. From this, the mean composition number average molecular weight of the polymer can then be determined GPC can also be used to determine the number average molecular weight, weight average molecular weight, and polydispersity. See John Keith Harris, Ph.D. Thesis entitled, “Characterization and manipulation of vesicles formed from 1,2-butylene oxide/ethylene oxide diblock copolymers,” Michigan State University, 2003, the entirety of which is incorporated herein by reference. It is also possible to use matrix-assisted laser desorption/ionization mass spectroscopy (MALDI) to determine the number of EO and BO units.

“Hydrophobic,” for purposes of this disclosure, refers to a component that is more soluble in dodecane than in water. Such components generally have a log octanol/water partition coefficient greater than 1. Examples may be found in the CRC Handbook of Chemistry & Physics, edited by D. R. Linde, CRC Press, Florida, 74th Ed. (1993-94), Sec. 16, page 24 et seq.

“Cosmetically acceptable” refers to ingredients typically used in personal care compositions, and is intended to underscore that materials that are toxic, irritating, or unpleasant smelling when present in the amounts typically found in personal care compositions are not contemplated as part of the present invention.

“Personal care compositions” are those that are to be topically (including mouth, ear, and nasal cavities) applied to a person. Examples of personal care compositions include skin care products (e.g., facial cream, moisturizers, leave on and rinse off lotions, sunscreens, foundation, mascara, eye-liner, lipstick, and the like), oral care products (such as toothpastes and rinses), nail care products (such as polish and conditioners), and hair care products (including leave on and rinse off conditioners, styling gels and hairsprays).

“Personal care actives” are components that impart a specific performance property to a personal care composition. Examples of personal care actives include typical actives for skin care products (e.g., facial cream, moisturizers, leave on and rinse off lotions, sunscreens, foundation, mascara, eye-liner, lipstick, and the like), oral care products (such as toothpastes and rinses), nail care products (such as polish and conditioners), and hair care products (including leave on and rinse off conditioners, styling gels and hairsprays). Examples of actives include antibacterial compounds (e.g. triclosan) in toothpaste, polypheols, flavinoids and isoflvinoids, coenzyme Q10 and derivatives thereof, carotene and derivatives thereof, salicylic acid and derivatives thereof, dehydroepiandrosterone (DHEA), hydrophobic polysaccharides, proteins, including enzymes and peptides, and botanicals. Examples of sunscreens include paraminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide and zinc oxide, diethanolamine methoxycinnamate, digalloy trioleate, ethyl dihydroxypropyl PABA, glyceryl aminobenzoate, lawsone with dihydroxy acetone, and red petrolatum. Vitamins include Vitamin A and esters thereof, Vitamin D and derivatives thereof, Vitamins B3 and B5 and derivatives thereof, Vitamin E and esters thereof, Vitamin F and derivatives thereof, and Vitamin K.

Dyes include liposoluble dyes, such as Sudan red, DC Red 17, DC Green 6, β-carotene, soybean oil, Sudan brown, DC Yellow 11, DC Violet 2, DC Orange 5, and quinoline yellow.

Flavors include flavor oils, such as peppermint, wintergreen, citrus, fruit, vanilla, and cinnamon. Most flavors are hydrophobic, and thus contemplated.

Fragrances include any component which provides a pleasant scent. Examples include scents that are floral, ambery, woody, leather, chypre, fougere, musk, vanilla, fruit, and/or citrus. Fragrances are often oils obtained by extraction of natural substances or synthetically produced. In one embodiment, the fragrance is one of the essential oils.

In a preferred embodiment, the hydrophobic, cosmetically acceptable, personal care active, vitamin, dye, flavor, or fragrance, is one that is susceptible to reaction or degradation in the personal care composition, including evaporation, photo-degradation, oxidation, or any other processes which leave such a component less potent or effective.

The hydrophobic, cosmetically acceptable, component is present in a range from about 1 ppm to about 50,000 ppm (5 weight percent by weight of the composition). For example, in many skin care applications, the hydrophobic, cosmetically acceptable, component is present in a range from about 10 ppm to about 10,000 ppm (1 weight percent by weight of the composition). Similarly, the invention includes hydrophobic, cosmetically acceptable, components present in a concentration greater than about 1 ppm, preferably greater than about 10 ppm, and less than about 50,000 ppm, preferably less than about 10,000 ppm.

In one embodiment, the weight ratio between the hydrophobic, cosmetically acceptable, component and the block polymer is from about 10:1 to about 1:100. In one embodiment, the weight ratio between the hydrophobic, cosmetically acceptable, component and the block polymer is about 1:3. In one embodiment, the weight ratio between the hydrophobic, cosmetically acceptable, component and the block polymer is about 1:1.

The block polymer may be selected based on a variety of considerations. For example, although the preferred range of EO units is about 4 to about 16 ethylene oxide units under normal conditions, the upper range can be extended by increasing the temperature or the salienty. Similarly, the BO block can be selected for predetermined conditions, for example, a longer BO block will result in increased stability for the resulting vesicle, but will require more energy to create the vesicle.

Methods of producing suitable block polymers, for example by anionic polymerization, are well known. In one embodiment, the block polymer may be a di-block polymer. U.S. Pat. No. 5,587,143, the entirety of which is incorporated herein by reference, discloses butylene oxide-ethylene oxide block copolymers. Likewise, the behavior of ethylene oxide/butylene oxide diblock copolymers in aqueous solutions is discussed in J. Keith Harris et al., “Spontaneous Generation of Multilamellar Vescicles from Ethylene Oxide/Butylene Oxide Diblock Copolymers”, Langmuir 2002, 18, 5337-5342, the entirety of which is incorporated herein by reference herein.

In one embodiment, the block polymer is a di-block polymer that has about 10 to about 13 EO units and about 10 to about 13 BO units. In one embodiment, the di-block polymer has an about equal number of EO units and BO units. In one embodiment, the di-block polymer has about 12 EO units and about 12 BO units. In another embodiment, the di-block polymer has about 11 EO units and about 12 BO units.

Alternatively, the block polymer may be a tri-block polymer. For systems where water is not the continuous phase (such as mineral oil or hydrocarbon media), the tri-block polymer can be BO-EO-BO. For aqueous based systems, the tri-block polymer can be EO-BO-EO. In a preferred embodiment, the initial EO block of the EO-BO-EO tri-block polymer is a block of polymerized ethylene oxide having about 4 to about 50 EO units.

Some personal care compositions of the present invention, such as antiperspirants, are contemplated to be waterless, or at least to contain negligible amounts of water. However, many other personal care compositions of the present invention do include water as an ingredient.

In some embodiments, water is present in a range from about 5 to about 99 weight percent by weight of the composition. In one embodiment, the composition is more than 20 weight percent water. In one embodiment, water is present in a range from about 20 to about 40 weight percent by weight of the composition. In other embodiments, the composition is more than 40 weight percent water. In one embodiment, water is present in a range from about 40 to about 60 weight percent by weight of the composition. In other embodiments, water is present in a range from about 60 to about 80 weight percent by weight of the composition. In one embodiment, the composition is more than 75 weight percent water. The recited ranges are given to amply illustrate certain features of the invention; however, additional ranges are understood to be contemplated. Indeed, all novel combinations and subcombinations found within the above ranges are contemplated and may be placed in the appended claims.

In one embodiment, the polydispersity of the block polymer is below about 1.5. In one embodiment, the polydispersity of the block polymer is below about 1.3. In one embodiment, the polydispersity of the block polymer is below about 1.15. In one embodiment, the polydispersity of the block polymer is about 1.

In some embodiments, the block polymer has a molecular weight of less than 2000 (weight average). In one embodiment, the block polymer has a molecular weight of less than 1400 (weight average).

The block polymer may be present in a range from about 0.1 to about 30 weight percent by weight of the composition. In some embodiments, the block polymer is present in a range from about 0.5 to about 2 weight percent by weight of the composition; preferably, the block polymer is present in a range from about 0.8 to about 1.2 weight percent by weight of the composition.

Other optional ingredients for personal care compositions of the present invention include cosmetically acceptable emollients, sunscreens, surfactants, emulsifiers, preservatives, rheology modifiers, colorants, preservatives, pH adjustors, propellants, reducing agents, fragrances, foaming agents, tanning agents, depilatory agents, flavors, astringents, antiseptics, deodorants, antiperspirants, insect repellants, bleaches, lighteners, anti-dandruff agents, adhesives, polishes, strengtheners, fillers, barrier materials, or biocides.

The moisturizers include 2-pyrrolidone-5-carboxylic acid and its salts and esters, alkyl glucose alkoxylates or their esters, fatty alcohols, fatty esters, glycols and, in particular, methyl glucose ethoxylates or propoxylates and their stearate esters, isopropyl myristate, lanolin or cetyl alcohols, aloe, silicones, propylene glycol, glycerol and sorbitol.

Conditioners include stearalkonium chloride, dicetyldimonium chloride, lauryl methyl gluceth-10 hydroxypropyldimonium chloride, and conditioning polymers such as polyquaternium-10, polyquaternium-24 and chitosan and derivatives thereof.

Examples of oils include hydrocarbon-based oils of animal origin, such as squalene, hydrocarbon-based oils of plant origin, such as liquid triglycerides of fatty acids comprising from 4 to 10 carbon atoms, for instance heptanoic or octanoic acid triglycerides, or alternatively, oils of plant origin, for example sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, MIGLYOL 810, 812 and 818 (from Dynamit

Nobel), synthetic esters and ethers, especially of fatty acids, for instance the oils of formulae R¹COOR² and R¹ OR² in which R¹ represents a fatty acid residue comprising from 8 to 29 carbon atoms and R² represents a branched or unbranched hydrocarbon-based chain comprising from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate, hydroxylated esters, for instance isostearyl lactate, octyl hydroxystearate, octyldodecyl hydroxystearate, diisostearyl malate, triisocetyl citrate and fatty alcohol heptanoates, octanoates and decanoates, polyol esters, for instance propylene glycol dioctanoate, neopentyl glycol diheptanoate and diethylene glycol diisononanoate, pentaerythritol esters, for instance pentaerythrityl tetraisostearate, lipophilic derivatives of amino acids, such as isopropyl lauroyl sarcosinate, such as is sold under the name ELDEW SL 205 (from Ajinomoto), linear or branched hydrocarbons of mineral or synthetic origin, such as mineral oils (mixtures of petroleum-derived hydrocarbon-based oils), volatile or non-volatile liquid paraffins, and derivatives thereof, petroleum jelly, polydecenes, isohexadecane, isododecane, hydrogenated isoparaffin (or polyisobutene), silicone oils, for instance volatile or non-volatile polymethylsiloxanes (PDMS) comprising a linear or cyclic silicone chain, which are liquid or pasty at room temperature, especially cyclopolydimethylsiloxanes (cyclomethicones) such as cyclopentasiloxane and cyclohexadimethylsiloxane, polydimethylsiloxanes comprising alkyl, alkoxy or phenyl groups, which are pendent or at the end of a silicone chain, these groups comprising from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes 2-phenylethyltrimethyl siloxysilicates and polymethylphenylsiloxanes, fluoro oils such as partially hydrocarbon-based and/or partially silicone-based fluoro oils, ethers such as dicaprylyl ether (CTFA name: dicaprylyl ether), and C₁₂-C₁₅ fatty alcohol benzoates (FINSOLV TN from Finetex), mixtures thereof.

Oils include mineral oil, lanolin oil, coconut oil and derivatives thereof, cocoa butter, olive oil, almond oil, macadamia nut oil, aloe extracts such as aloe vera lipoquinone, jojoba oils, safflower oil, corn oil, liquid lanolin, cottonseed oil, peanut oil, hydrogenated vegetable oil, squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, and the oil of cereal germs.

Other suitable emollients include dicaprylyl ether, C₁₂₋₁₅ alkyl benzoate, DC 200 FLUID 350 silicone fluid (from Dow Corning Corp.), isopropyl palmitate, octyl palmitate, isopropyl myristate, hexadecyl stearate, butyl stearate, decyl oleate, acetyl glycerides, the octanoates and benzoates of C₁₂₋₁₅ alcohols, the octanoates and decanoates of alcohols and polyalcohols such as those of glycol and glyceryl, ricinoleates esters such as isopropyl adipate, hexyl laurate and octyl dodecanoate, dicaprylyl maleate, phenyltrimethicone, and aloe vera extract. Solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate.

In some embodiments, the personal care composition further comprises an optional rheology modifier as a thickener. Examples of thickeners include polymers, for example, modified or unmodified carboxyvinyl polymers, such as the products sold under the names CARBOPOL and PEMULEN (INCI name: Acrylates/C₁₀₋₃₀ alkyl acrylate crosspolymer; available from Noveon), polyacrylates and polymethacrylates, such as the products sold under the names LUBRAJEL and NORGEL (from Guardian) or HISPAGEL (from Hispano Chimica), polyacrylamides, 2-acrylamido-2-methylpropanesulfonic acid polymers and polymers, which are optionally crosslinked and/or neutralized, for instance the poly(2-acrylamido-2-methylpropane-sulfonic acid) sold by Clariant (INCI name: ammonium polyacryldimethyltauramide), emulsified crosslinked anionic polymers of acrylamide and AMPS, such as those sold under the name SEPIGEL 305 (INCI name: Polyacrylamide/C13-14 Isoparaffin/Laureth-7; from Seppic) and under the name SIMULGEL 600 (INCI name: Acrylamide/Sodium acryloyldimethyltaurate polymer/Isohexadecane/Polysorbate 80; from Seppic), polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethylcellulose, hydroxymethylcellulose and hydroxypropylcellulose, associative polymers, for instance associative polyurethanes, polymers comprising at least two hydrocarbon-based lipophilic chains comprising from 6 to 30 carbon atoms, separated with a hydrophilic sequence, such as the polyurethanes sold under the names SERAD FX1010, SERAD FX1100 and SERAD FX1035 (from Hüls America), RHEOLATE 255, RHEOLATE 278 and RHEOLATE 244 (INCI name: Polyether-urea-polyurethane; from Rheox), DW 1206F, DW 1206J, DW 1206B, DW 1206G, and ACRYSOL RM 2020 (from Röhm & Haas).

Colorants include pigments, which are used especially in make-up, including metal oxide pigments, titanium dioxide, optionally surface-treated, zirconium oxide or cerium oxide, zinc oxide, iron oxide (black, yellow or red), chromium oxide, manganese violet, ultramarine blue, chromium hydrate and ferric blue, carbon black, pigments of barium, strontium, calcium or aluminum (for example D&C or FD&C), cochineal carmine, mica coated with titanium or with bismuth oxychloride, titanium mica with iron oxides, titanium mica with, especially, ferric blue or chromium oxide, titanium mica with an organic pigment, nacreous pigments based on bismuth oxychloride, goniochromatic pigments, for example pigments with a multilayer interference structure, reflective pigments, for example particles with a silver-coated glass substrate, glass substrate coated with nickel/chromium/molybdenum alloy, glass substrate coated with brown iron oxide, particles comprising a stack of at least two polymer layers, for instance MIRROR GLITTER (from 3M).

Dyes include water-soluble dyes such as copper sulfate, iron sulfate, water-soluble sulfopolyesters, rhodamines, natural dyes, for instance carotene and beetroot juice, methylene blue, caramel, the disodium salt of tartrazine and the disodium salt of fuschin, and mixtures thereof. Liposoluble dyes from the list above may also optionally be used.

Preservatives include alcohols, aldehydes, methylchloroisothiazolinone and methylisothiazolinone, p-hydroxybenzoates, and in particular methylparaben, propylparaben, glutaraldehyde and ethyl alcohol.

The pH adjustors include inorganic and organic acids and bases and in particular aqueous ammonia, citric acid, phosphoric acid, acetic acid, and sodium hydroxide.

Reducing agents include ammonium thioglycolate, hydroquinone and sodium thioglycolate.

Fragrances may be aldehydes, ketones, or oils obtained by extraction of natural substances or synthetically produced as described above. Often, fragrances are accompanied by auxiliary materials, such as fixatives, extenders, stabilizers and solvents.

Biocides include antimicrobials, bactericides, fungicides, algaecides, mildicides, disinfectants, antiseptics, and insecticides.

The amount of optional ingredients effective for achieving the desired property provided by such ingredients can be readily determined by one skilled in the art.

In one embodiment, the present invention provides a personal care composition, comprising a hydrophobic, cosmetically acceptable, component comprising at least one of a personal care active, vitamin, dye, flavor, or fragrance; a block polymer comprising at least one block of polymerized ethylene oxide having about 4 to about 16 ethylene oxide units and at least one block of a polymerized butylene oxide having about 4 to about 40 butylene oxide units; and water, wherein the hydrophobic, cosmetically acceptable, component is present when the block polymer and water are combined. In a preferred embodiment, the hydrophobic, cosmetically acceptable component contacts the block polymer prior to addition of the water.

In another embodiment, the present invention provides a process for preparing the personal care composition, comprising blending the hydrophobic, cosmetically acceptable, component with the block polymer in the absence of organic solvents; adding the aqueous phase and then, agitating the mixture. In one embodiment, the agitation includes gently shaking the container holding the blend. The blend may then be diluted and the remaining components added. The process can be performed at room temperature.

In another embodiment, the present invention provides methods for stabilizing a degradable or reactive hydrophobic, cosmetically acceptable, component in an aqueous composition, comprising blending the hydrophobic component with a block polymer having about 4 to about 16 EO units and about 4 to about 40 BO units, thereby encapsulating a percentage of the hydrophobic component in block polymer.

### Examples

The following examples are for illustrative purposes only and are not intended to limit the scope of the present invention. All percentages are by weight unless otherwise specified.

### Example 1

Exemplary personal care compositions contain the components recited in TABLE 1.

TABLE 1

| | Batch 1 | Batch 2 |
|---|---|---|
| A | 1000 ppm Vitamin A | — |
| | 1% EO (11)/BO (12) | 1% EO (11)/BO (12) |
| | block polymer | block polymer |
| | Balance water | Balance water |
| B | — | 1000 ppm Vitamin A |

For Batch 1, the hydrophobic component (Vitamin A), block polymer, and water are added together and gently shaken.

For Batch 2, the block polymer and water are added together and gently shaken. Then the hydrophobic component (Vitamin A) is added.

### Example 2

Formulations made substantially according to the protocol described above in Example 1 were placed in a 50° C. oven for six weeks. The initial samples rated a 1 on the Gardner color test (ASTM D1544). After six weeks, Batch 1 showed a 2 on the Gardner scale, whereas Batch 2 showed a 7, indicating significant degradation. While not intending to be bound by theory, it is believed that the block polymer forms vesicles in the aqueous solution, thereby encapsulating a portion of the aqueous solution. When Vitamin A is present in the aqueous solution, it too is encapsulated and is accordingly protected from degradation. If the Vitamin A is added after encapsulation, as with Batch 2, then substantially less Vitamin A is protected. Accordingly, Batch 1 is a more preferred embodiment.

### Example 3

Exemplary personal care compositions contain the components recited in TABLE 2.

TABLE 2

| Batch 3 (Comparative) | Batch 4 |
|---|---|
| 1000 ppm Vitamin A | 1000 ppm Vitamin A |
| 1% EO (25)/BO (36) | 1% EO (11)/BO (12) |
| block polymer | block polymer |
| MW = 3672 g/mole | MW = about 1400 g/mole |
| Balance water | Balance water |

For both batches, the hydrophobic component (Vitamin A), block polymer, and water are added together and gently shaken.

### Example 4

Formulations made substantially according to the protocol described above in Example 3 were placed in a 50° C. oven. Initially, Batch 3 rated a 1.5 on the Gardner color test (ASTM D1544) and Batch 4 rated a 1. After six weeks, Batch 4 showed a 2 on the Gardner scale. In contrast, Batch 3 showed a 6 on the Gardner scale after three weeks, indicating significant degradation. While not intending to be bound by theory, it is believed that the Batch 3 failed to form as many vesicles in the aqueous solution, and thus, Vitamin A was not as protected from degradation.

Regarding the molecular weight of Batch 4, GPC determined the molecular weight to be 1263 g/mol. NMR analysis gave 12 BO/11 EO. Using the recipe quantities, the ideal molecular weight for methanol+12 mol BO+11 mol EO would be 1352 g/mol (32+(11×72)+(12×44)). The actual molecular weight is believed to be 1396 g/mole. The molecular weight from OH number titration (1.16% OH) was 1465 g/mol.

It is understood that the present invention is not limited to the embodiments specifically disclosed and exemplified herein. Various modifications of the invention will be apparent to those skilled in the art. Such changes and modifications may be made without departing from the scope of the appended claims.

Moreover, each recited range includes all combinations and subcombinations of ranges, as well as specific numerals contained therein. Additionally, the disclosures of each patent, patent application, and publication cited or described in this document are hereby incorporated herein by reference, in their entireties.

1. A personal care composition, comprising:
a hydrophobic, cosmetically acceptable, component comprising at least one of a personal care active, vitamin, dye, flavor, or fragrance; and
a block polymer comprising at least one block of polymerized ethylene oxide having about 4 to about 16 ethylene oxide units and at least one block of a polymerized butylene oxide having about 4 to about 40 butylene oxide units.

2. The personal care composition of claim 1, further comprising water, wherein the hydrophobic, cosmetically acceptable, component is present when the block polymer and water are combined.

3. The personal care composition of claim 2, wherein the composition is more than 20 weight percent water.

4. The personal care composition of claim 2, wherein the composition is more than 40 weight percent water.

5. The personal care composition of claim 2, wherein the composition is more than 75 weight percent water.

6. The personal care composition of claim 2, wherein the water is present in a range from about 5 to about 99 weight percent by weight of the composition.

7. (canceled)

8. The personal care composition of claim 1, wherein the block polymer is a di-block polymer having about 10 to about 13 ethylene oxide units and about 10 to about 13 butylene oxide units.

9. The personal care composition of claim 1, wherein the block polymer is a di-block polymer having about 12 ethylene oxide units and about 12 butylene oxide units.

10. The personal care composition of claim 1, wherein the block polymer is a di-block polymer having about 11 ethylene oxide units and about 12 butylene oxide units.

11. The personal care composition of claim 1, wherein the polydispersity of the block polymer is below about 1.5.

12. The personal care composition of claim 1, wherein the polydispersity of the block polymer is below about 1.3.

13. The personal care composition of claim 1, wherein the polydispersity of the block polymer is below about 1.15.

14. The personal care composition of claim 1, wherein the block polymer has a weight average molecular weight of less than 2000.

15. The personal care composition of claim 1, wherein the block polymer has a weight average molecular weight of less than 1400.

16. The personal care composition of claim 1, wherein the block polymer is present in a range from about 0.1 to about 20 weight percent by weight of the composition.

17. The personal care composition of claim 1, wherein the block polymer is present in a range from about 0.5 to about 2 weight percent by weight of the composition.

18. The personal care composition of claim 1, wherein the block polymer is present in a range from about 0.8 to about 1.2 weight percent by weight of the composition.

19. The personal care composition of claim 1, wherein the hydrophobic, cosmetically acceptable, component is present in a range from about 1 ppm to about 50,000 ppm by weight of the composition.

20.-21. (canceled)

22. A process for preparing the personal care composition of claim 2, comprising:
blending the hydrophobic, cosmetically acceptable, component with the block polymer in the absence of organic solvents; and then, agitating.

23. A method for stabilizing a degradable or reactive hydrophobic, cosmetically acceptable, component in an aqueous composition comprising:
encapsulating a percentage of the hydrophobic component with a block polymer having at least one ethylene oxide block and at least one butylene oxide block.
